(19) United States
(12) Patent Application Publication
(10) Pub. No.: US 2024/0261362 A1
(43) Pub. Date: Aug. 8, 2024

(54) Use Of Vetiver Product In Preparation Of Drug Having Anti-Inflammatory Effect And Capable Of Improving Animal Immunity

(71) Applicant: Henan Agricultural University, Zhengzhou (CN)

(72) Inventors: Hongying Zhang, Zhengzhou (CN); Xiaoya Hao, Zhengzhou (CN); Liying Chen, Zhengzhou (CN); Mingfan Yang, Zhengzhou (CN); Yue Jin, Zhengzhou (CN); Xinsheng Li, Zhengzhou (CN)

(73) Assignee: Henan Agricultural University, Zhengzhou (CN)

(21) Appl. No.: 18/446,360

(22) Filed: Aug. 8, 2023

(30) Foreign Application Priority Data
Feb. 8, 2023 (CN) CN2023100872095

(51) Int. Cl. A61K 36/899; A61P 29/00

(52) U.S. Cl. CPC A61K 36/899; A61P 29/00; A61K 2236/331; A61K 2236/37; A61K 2236/39; A61K 2236/53

(57) ABSTRACT

The present disclosure belongs to the technical field of animal medicine, and in particular relates to use of a vetiver (Chrysopogon zizanioides) product in preparation of a drug having an anti-inflammatory effect and capable of improving animal immunity. In the present disclosure, the vetiver, especially a water extract of vetiver prepared from a root of the vetiver may improve an immune regulation function of the body. In this way, the expression of various inflammatory factors in the body may be alleviated to significantly enhance the animal immunity and the anti-inflammation. Moreover, the drug of the present disclosure has wide selection of raw materials and a low cost.

### CROSS REFERENCE TO RELATED APPLICATION

This application claims the priority of Chinese Patent Application No. 2023100872095, entitled “use of vetiver product in preparation of drug having anti-inflammatory effect and capable of improving animal immunity” filed with the China National Intellectual Property Administration on Feb. 8, 2023 which is incorporated herein by reference in its entirety.

### REFERENCE TO SEQUENCE LISTING

A computer readable XML file entitled “SEQUENCE LISTING.XML”, that was created on Jul. 11, 2023, with a file size of about 9727 bytes, contains the sequence listing for this application, has been filed with this application, and is hereby incorporated by reference in its entirety.

### TECHNICAL FIELD

The present disclosure belongs to the technical field of veterinary medicine, and in particular relates to use of a vetiver (Chrysopogon zizanioides) product in preparation of a drug having an anti-inflammatory effect and capable of improving animal immunity.

### BACKGROUND

With the development of animal husbandry in China, domesticated animals are affected by various adverse factors, which may decrease the immunity of the animal body, and thus cause diseases. The immune functions of animals are conducted by various immune cells and immune molecules, and their quantities and activities determine the strength of immunity. The immunity is one of the key factor of animal defense, and therefore, it has become the focus of research in animal breeding how to stimulate the immune system so as to to improve the animal immunity.

Inflammation is generally caused by many factors, such as physical elements, oxidative stress, microorganisms, and high-density feeding. Inflammation is an innate immune defense mechanism, and a part of the complex biological responses of the body tissues to noxious stimuli. Inflammation is a protective response of the immune system through which the adverse factors that cause cell and body damages are eliminated, and cell and tissue damages repaired. However, inflammation might in some cases be excessively aroused, leading to acute or chronic inflammatory responses and subsequent tissue/organ damages.

At present, chemically synthesized drugs or hormones are the main way to solve the excessive inflammatory responses. However, these substances generally have various toxic and side effects, such as the generation of resistant bacteria and harmful residues in animal products. Following the era of “reducing the use of antibiotics” and “using alternatives of antibiotics”, Chinese herbal medicine products have becoming widely used in the livestock and poultry industry. Chinese herbs have also been shown to enhance the immune function of livestock, thereby strengthening the body's defense. However, production costs of veterinary herbal drugs are usually high and medicinal resources limited.

### SUMMARY

A purpose of the present disclosure is to provide use of a vetiver (Chrysopogon zizanioides) product in preparation of a drug for improving animal immunity or preparation of a drug which possesses anti-inflammatory effect and improve animal immunity. Apart from their significant immunity-boosting potential, vetiver derivatives have the advantages of being readily available and low costs.

The present disclosure provides use of a vetiver product in preparation of a drug with the following effects of I or II:
I: improving animal immunity; and
II: enhancing animal immunity and anti-inflammation.

In one embodiment, the vetiver product includes a water extract of vetiver.

The present disclosure further provides a preparation method of a water extract of vetiver, including the following steps:
immersing the roots of vetiver in water to obtain a immersed solid-liquid mixture, decocting the immersed solid-liquid mixture, and separating the solid and liquid to obtain the water extract of vetiver.

In one embodiment, the decocting is conducted 1 to 3 times.

In one embodiment, during the decocting, the roots of vetiver and the water are at a mass-to-volume ratio of 1 g:(10-20) mL.

In one embodiment, the decocting is conducted at 90° C. to 100° C. for 30 min to 60 min.

The present disclosure further provides a water extract of vetiver prepared by the preparation method.

The present disclosure further provides a drug having an anti-inflammatory effect and capable of improving animal immunity, including the water extract of vetiver.

In one embodiment, the water extract of vetiver has a concentration of 0.5 g/mL to 2 g/mL.

In one embodiment, the drug has the water extract of vetiver with a mass percentage of 1.9% to 100%.

The present disclosure has the following beneficial effects:

The present disclosure provides a use of a vetiver product in the manufacture of a medicament for enhancing animal immunity or for manufacturing a medicament having anti-inflammatory activity capable of enhancing animal immunity. In the present disclosure, the vetiver, especially the water extract prepared from vetiver roots, can improve the immune regulation function and reduce the expression of inflammatory cytokines, thereby significantly improving the immunity and anti-inflammatory performance of animals. Moreover, the raw materials are easy to get and the cost is low.

### BRIEF DESCRIPTION OF THE DRAWINGS

To describe the technical solutions in the examples of the present disclosure or in the prior art more clearly, the accompanying drawings required for the examples will be briefly interpreted below.

FIG. 1 shows the proliferation rates of murine mononuclear macrophages in a vetiver experimental group in Use Example 1;

FIG. 2 shows the proliferation rates of murine mononuclear macrophages in a Radix Astragali control group in Use Example 1;

FIG. 3 shows the phagocytic index of murine mononuclear macrophages in a vetiver experimental group in Use Example 2;

FIG. 4 shows the phagocytic index of murine mononuclear macrophages in a Radix Astragali control group in Use Example 2;

FIG. 5 shows the NO secretion of mouse mononuclear macrophages in a vetiver experimental group in Use Example 3;

FIG. 6 shows the NO secretion of mouse mononuclear macrophages in a Radix Astragali control group in Use Example 3;

FIG. 7 shows the expression levels of iNOS mRNA of mouse mononuclear macrophages in the vetiver experimental group in Use Example 3;

FIG. 8 shows the expression levels of iNOS mRNA of mouse mononuclear macrophages in the Radix Astragali control group in Use Example 3;

FIG. 9 shows the expression levels of TNF-α mRNA of mouse mononuclear macrophages in a vetiver experimental group in Use Example 4;

FIG. 10 shows the expression levels of TNF-α mRNA of mouse mononuclear macrophages in a Radix Astragali control group in Use Example 4;

FIG. 11 shows the expression levels of IL-6 mRNA of mouse mononuclear macrophages in the vetiver experimental group in Use Example 4;

FIG. 12 shows the expression levels of IL-6 mRNA of mouse mononuclear macrophages in the Radix Astragali control group in Use Example 4;

FIG. 13 shows the expression levels of IL-1β mRNA of mouse mononuclear macrophages in the vetiver experimental group in Use Example 4;

FIG. 14 shows the expression levels of IL-1β mRNA of mouse mononuclear macrophages in the Radix Astragali control group in Use Example 4;

FIG. 15 shows the expression levels of TNF-α mRNA in mouse ear tissues of each experimental group in Use Example 8;

FIG. 16 shows the expression levels of IL-6 mRNA in the mouse ear tissues of each experimental group in Use Example 8; and

FIG. 17 shows the expression levels of IL-1β mRNA in the mouse ear tissues of each experimental group in Use Example 8.

### DETAILED DESCRIPTION OF THE EMBODIMENTS

The present disclosure provides use of a vetiver product in preparation of a drug with the following effects of I or II:
I: improving animal immunity; and
II: enhancing animal immunity and anti-inflammation.

In the present disclosure, the drug includes preferably a drug capable of improving animal immunity and anti-inflammation. The vetiver product includes preferably a water extract of vetiver, more preferably a root extract of the vetiver.

In the present disclosure, the drug prepared from the vetiver may significantly improve an immune regulation function of animal cells, such as increasing an activity of mononuclear macrophages, increasing a phagocytic ability of mononuclear macrophages, and reducing a secretion of NO and an expression level of iNOS in mononuclear macrophages. Meanwhile, the drug may alleviate the inflammation of body, such as reducing the expression levels of inflammatory factors including TNF-α, IL-1β, and IL-6.

The present disclosure further provides a preparation method of a water extract of vetiver, including the following steps:
immersing a root of vetiver into water to obtain an immersed solid-liquid mixture, decocting the immersed solid-liquid mixture, and conducting solid-liquid separation to obtain the water extract of vetiver.

In the present disclosure, the root of vetiver is preferably cut to obtain root pieces of the vetiver. There is no special limitation on a cutting method and a size of the root pieces of the vetiver, and the root of vetiver may be cut by a conventional cutting method in the art.

In the present disclosure, the root pieces of the vetiver are immersed into water. The root pieces of the vetiver and the water are at a mass-to-volume ratio of preferably 1 g:(10-20) mL, more preferably 1 g:(12-18) mL, and even more preferably 1 g: 15 mL. The immersing is conducted for preferably 2 h to 4 h, more preferably 3 h.

In the present disclosure, an obtained immersed solid-liquid mixture is decocted to obtain a water extract of vetiver. The decocting is conducted preferably 1 to 3 times, more preferably 2 times. When the decocting is conducted for 2 to 3 times, the filtrate is preferably consisted of filtrates after multiple decoctions. That is, the solid-liquid mixture is subjected to first decocting to obtain a filtrate I, and an obtained filter residue is mixed with water to conduct second decocting to obtain a filtrate II, and then filtrate III. The filtrates I to III are merged to be the above-mentioned filtrate.

In the present disclosure, during the decocting, the mass-to-volume ratio of the root of vetiver or the decocted vetiver root filter residue and water is preferably 1 g:(10-20) mL, more preferably 1 g: 15 mL. The decocting is conducted at preferably 90° C. to 100° C., more preferably 100° C. for preferably 30 min to 60 min, more preferably 45 min to 60 min, and even more preferably 60 min.

The present disclosure further provides a water extract of vetiver produced by the abovementioned preparation scheme.

The present disclosure further provides a drug having an anti-inflammatory effect and immunity improving capacity, including the water extract of vetiver. The drug is preferably an aqueous vetiver solution or a composition containing the water extract of vetiver, with a concentration of preferably 0.5 g/mL to 2 g/mL, and more preferably 1 g/mL. The preferable drug forms include powder and oral aqua, more preferably oral aqua. The mass percentage of the water extract of vetiver in the present drug is preferably 1.9% to 100%, more preferably 1% to 20%, and even more preferably 7%.

The present disclosure further provides a preparation scheme of the drug, including the following steps: subjecting the water extract of vetiver to vacuum concentration, centrifugation, and microfiltration to obtain the drug. The vacuum concentration is conducted at preferably 75° C. to 85° C. The centrifugation is conducted at preferably 12,000 rpm for preferably 30 min. The microfiltration is conducted using a filter membrane with a pore size of preferably 0.22 μm to 0.45 μm, more preferably 0.22 μm.

In the present disclosure, the drug may be used in the form of feed additive to improve the animal immunity and inflammation resistance. Based on the dosage conversion between various animals, the drug in mice is at a dosage of preferably 3.7 g/kg. By referring to “Conversion Method of Drug Dosage Between Human and Animals and Between Various Animals [EB/OL]. (2012-4-1)” (Animal Experiment Center of Ningxia Medical University), the preferred drug dosages can be computed as: 1.333 g/kg in rabbits, 1.08 g/kg in cats, and 0.747 g/kg in dogs.

In order to further illustrate the present disclosure, the technical solutions provided by the present disclosure will be described in detail below in conjunction with accompanying drawings and examples, but they should not be construed as limiting the protection scope of the present disclosure.

### Example 1

A preparation method of a water extract of vetiver included the following steps:

A certain mass of dried vetiver root was accurately weighed with an electronic balance, cut into pieces, added with 15 times the amount of water, and immersed for 3 h. A resulting mixture was boiled on low heat for 60 min, and a filtrate I was collected. 15 times the amount of water was added to an obtained filter residue, boiled on low heat for 60 min, and a filtrate II was collected. The 2 filtrates were combined, centrifuged to obtain a supernatant, and the supernatant was filtered through a 0.22 μm filter membrane to obtain the water extract of vetiver.

### Example 2

A drug having an anti-inflammatory effect and capable of improving animal immunity included the following components:

The water extract of vetiver prepared in Example 1 was subjected to vacuum concentration to obtain an aqueous vetiver solution with a concentration of 1 g/mL. The aqueous vetiver solution with a concentration of 1 g/mL was centrifuged, and an obtained supernatant was filtered through a 0.22 μm filter membrane to obtain the drug having an anti-inflammatory effect and capable of improving animal immunity, which was recorded as an experimental drug.

### Comparative Example 1

A control drug was prepared according to the steps in Example 1 and Example 2, with a difference that: the dried root of vetiver in Example 1 was replaced by Radix Astragali. In this field, Radix Astragali and its extracts have the functions of immune regulation, anti-inflammation, and anti-oxidation. Accordingly, the traditional Chinese medicine Radix Astragali was selected as a positive control.

### Use Example 1

### The Influences of the Drugs in Example 2 and Comparative Example 1 to Cell Activity of Mouse Mononuclear Macrophages

Macrophages RAW 264.7 (1×10⁵ cells/mL) in the logarithmic growth phase were added to a 96-well plate, and 100 μL of a cell suspension was added to each well. The cell suspension included the RAW 264.7 cells and a cell medium, and the cell medium was a high-sugar DMEM medium containing 10% fetal bovine serum. After culturing for 12 h, a cell supernatant was discarded, and a remaining product was washed 2 to 3 times with PBS (pH=7.4). The drug was divided into high, medium, and low doses.

The vetiver experimental groups and the Radix Astragali control groups were set up: the vetiver experimental groups and the Radix Astragali control groups each included 8 groups. The specific grouping was as follows:

Vetiver experimental group: the experimental drug prepared in Example 2 was diluted with a maintenance solution (2% FBS and DMEM medium) to obtain 62.5 μg/mL, 125 μg/mL, and 250 μg/mL of water extract of vetivers.

Blank control group (C group); LPS group: 1 μg/mL lipopolysaccharide; H+LPS group: 250 μg/mL water extract of vetiver and 1 μg/mL lipopolysaccharide; M+LPS treatment group: 125 μg/mL water extract of vetiver and 1 μg/mL lipopolysaccharide; L+LPS treatment group: 62.5 μg/mL water extract of vetiver and 1 μg/mL lipopolysaccharide; H group: 250 μg/mL water extract of vetiver; M group: 125 μg/mL water extract of vetiver; and L group: 62.5 μg/mL water extract of vetiver.

Radix Astragali control group: the control drug prepared in Comparative Example 1 was diluted with the maintenance solution (2% FBS and DMEM medium) to obtain 62.5 μg/mL, 125 μg/mL, and 250 μg/mL of Radix Astragali water extracts.

Blank control group (C group); LPS group: 1 μg/mL lipopolysaccharide; H+LPS group: 250 μg/mL Radix Astragali water extract and 1 μg/mL lipopolysaccharide; M+LPS treatment group: 125 μg/mL Radix Astragali water extract and 1 μg/mL lipopolysaccharide; L+LPS treatment group: 62.5 μg/mL Radix Astragali water extract and 1 μg/mL lipopolysaccharide; H group: 250 μg/mL Radix Astragali water extract; M group: 125 μg/mL Radix Astragali water extract; and L group: 62.5 μg/mL Radix Astragali water extract.

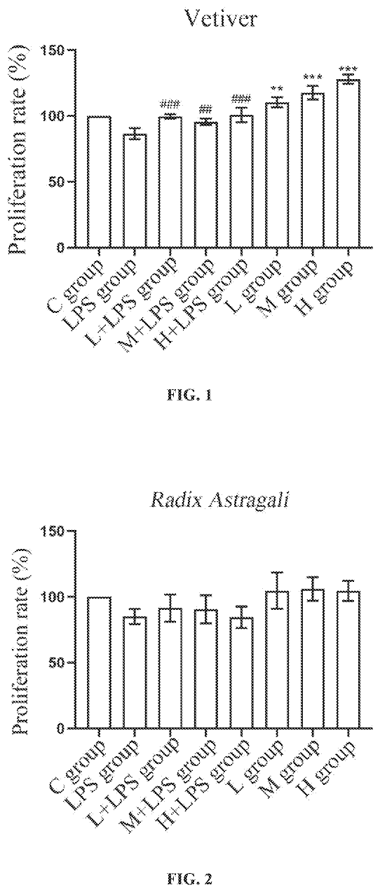

Each diluted drug was added to a 96-well plate, at 100 μL per well with 5 replicate wells in each group, and incubated in a CO₂ incubator at 37° C. for 48 h. 2 h before the end of the culture, 10 μL of a CCK-8 solution was added to each well, and the culture was continued for 2 h, and an OD value was measured at 450 nm. According to the OD value as an influence of the mouse mononuclear macrophage activity induced by LPS in vitro of the present disclosure, a cell proliferation rate was calculated according to the following formula, and an average of 5 duplicate wells was taken. The results were shown in FIG. 1, FIG. 2 and Table 1.

$$Cell proliferation rate ( \% ) = ( {OD}_{objective} - {OD}_{drug self control} ) / {OD}_{cell control group} \times 100 \% .$$

The OD_{objective} was the OD value of the experimental group except the C group; the OD_{drug self control} was the detection of the drug itself in order to reduce the error during the test, that is, the liquid of each concentration after the drug is diluted; and the OD_{cell control group} was the OD value of the C group.

TABLE 1
Influence of experimental drug in Example 2 and
control drug in Comparative Example 1 on activity
of mouse mononuclear macrophages (%)

| | Vetiver | Radix Astragali |
|---|---|---|
| Grouping | experimental group | control group |
| C group | 100 | 100 |
| LPS group | 86.73 ± 4.22 | 85.79 ± 8.73 |
| L + LPS group | 99.71 ± 1.71^{###} | 91.51 ± 10.35 |
| M + LPS group | 95.66 ± 2.39^{##} | 90.58 ± 10.62 |
| H + LPS group | 100.77 ± 5.49^{###} | 87.03 ± 5.66 |
| L group | 110.47 ± 3.81** | 109.94 ± 10.92 |
| M group | 117.69 ± 5.18*** | 110.36 ± 5.29 |
| H group | 127.98 ± 3.57*** | 105.81 ± 5.24 |

Notes:
compared with the blank control group (C group),
* P < 0.05,
**P < 0.01,
***P < 0.001; compared with the LPS group,
^{#} P < 0.05,
^{##}P < 0.01,
^{###}P < 0.001; the same below, no more detailed description.

Based on Table 1 and FIG. 1 to FIG. 2, it is concluded that in the vetiver experimental group, 62.5 μg/mL, 125 μg/mL, and 250 μg/mL of the water extract of vetivers acted on the mouse mononuclear macrophages alone or together with lipopolysaccharide (LPS) on the mouse mononuclear macrophages, and can significantly increase the proliferation rate of the mouse mononuclear macrophages, and the proliferation effect is better than that of the Radix Astragali control group. It shows that the experimental drug in Example 2 of the present disclosure can improve the activity of mouse mononuclear macrophages.

### Use Example 2

### The Influence of the Drugs in Example 2 and Comparative Example 1 on a Phagocytic Ability of Mouse Mononuclear Macrophages

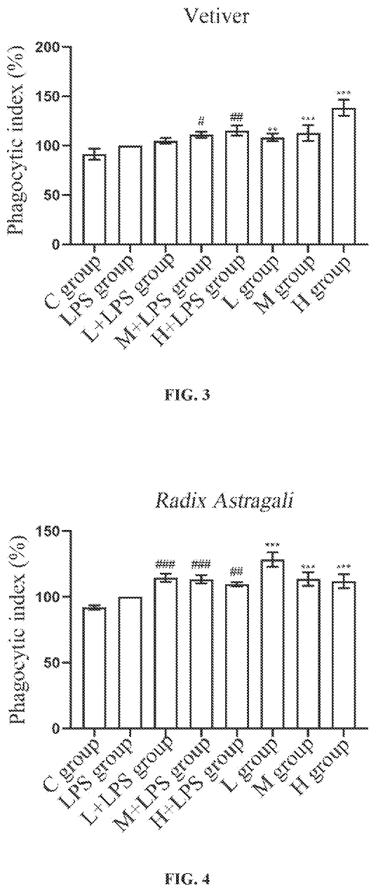

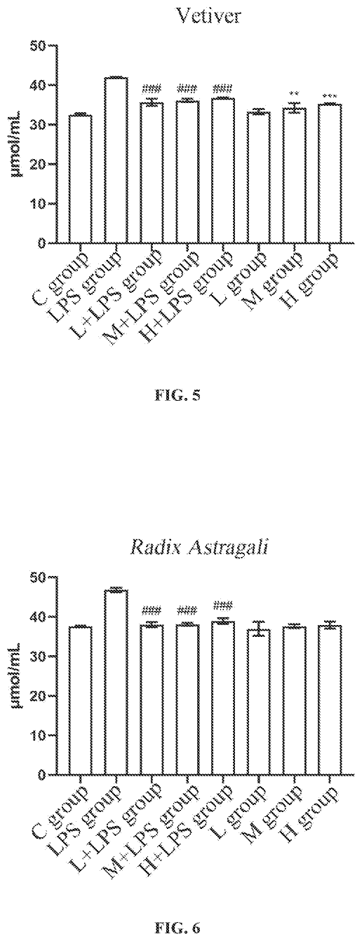

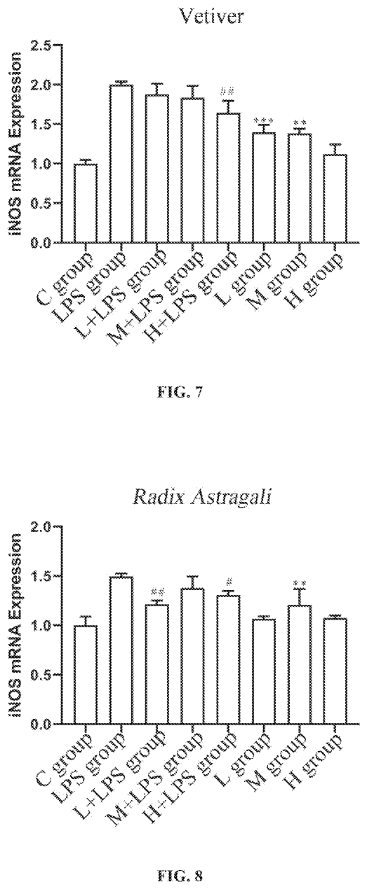

Macrophages RAW 264.7 (1×10⁵ cells/mL) in the logarithmic growth phase were inoculated into a 96-well plate, and 100 μL of a cell suspension was added to each well. The cell suspension included the RAW 264.7 cells and a cell medium, and the cell medium was a high-sugar DMEM medium containing 10% fetal bovine serum. After culturing for 12 h, the groups were set up and treated with drugs in the same way as Use Example 1. After 48 h, a supernatant was discarded, and a 0.05% neutral red solution was added for staining. After 1 h, the neutral red solution was discarded, and a remaining product was washed 3 times with PBS, and 150 μL of a cell lysate (including ethanol and acetic acid at a volume ratio of 1:1) was added to each well, shaken and mixed for 15 min, and an OD value was measured at 550 nm. The phagocytic ability of macrophages RAW 264.7 on neutral red was expressed by phagocytic index (PI), and the results were shown in Table 2 and FIG. 3 to FIG. 4.

$$PI ( \% ) = ( {A}_{S} / {A}_{I} ) \times 100 ;$$

where
the A_{S} was an absorbance of the experimental group (including cells, medium, and drug); and
the A₁ was an absorbance of the LPS group (including cells, LPS, and medium).

TABLE 2
Influence of experimental drug in Example 2 and
control drug in Comparative Example 1 on phagocytic
ability of mouse mononuclear macrophages (%)

| | Vetiver | Radix Astragali |
|---|---|---|
| Grouping | experimental group | control group |
| C group | 91.26 ± 5.58 | 92.05 ± 1.64 |
| LPS group | 100 | 100 |
| L + LPS group | 104.98 ± 2.93 | 114.54 ± 3.16^{###} |
| M + LPS group | 111.04 ± 3.11^{#} | 113.34 ± 3.04^{###} |
| H + LPS group | 115.26 ± 5.07^{##} | 109.56 ± 1.56^{##} |
| L group | 108.33 ± 3.90** | 128.35 ± 5.53*** |
| M group | 112.81 ± 8.03*** | 113.58 ± 5.12*** |
| H group | 138.35 ± 8.31*** | 111.95 ± 5.22*** |

From Table 2 and FIG. 3 to FIG. 4, it is concluded that in the present disclosure, the 3 different concentrations of the water extract of vetivers of the vetiver experimental group, or the 3 different concentrations of the Radix Astragali water extracts of the Radix Astragali control group all act on macrophages alone or acts on macrophages together with LPS. The results prove that the above water extracts all can significantly improve the phagocytic ability of the mouse mononuclear macrophages, and the water extract of vetivers and the Radix Astragali water extracts present similar effects, both significantly higher than those of the LPS group. This further shows that the experimental drug in Example 2 of the present disclosure can improve the activity of mouse mononuclear macrophages.

### Use Example 3

The Influence of the Drugs in Example 2 and Comparative Example 1 on the NO Secretion and iNOS Expression of Mouse Mononuclear Macrophages

RAW 264.7 macrophages in a well growth state were selected, a cell suspension with a cell concentration adjusted to 5×10⁵ cells/mL (the cell suspension included the RAW 264.7 cells and a cell medium, and the cell medium was a high-sugar DMEM medium containing 10% fetal bovine serum) was added, and the cells were inoculated in a 12-well culture plate, at 1 mL per well. After culturing for 12 h, the experimental grouping and drug treatment were conducted in a same manner as that of Use Example 1. After continuing to culture for 48 h, a supernatant was taken, and a level of cellular NO production was detected with the instructions of an NO kit (Beyotime; product number S0021S). A detection method and detection primers of iNOS gene referred to those of Use Example 4, and the detection results were shown in Table 3 and FIG. 5 to FIG. 8.

TABLE 3
Influence of experimental drug in Example 2 and
control drug in Comparative Example 1 on NO
of mouse mononuclear macrophages (μmol/mL)

| | Vetiver | Radix Astragali |
|---|---|---|
| Grouping | experimental group | control group |
| C group | 32.58 ± 0.28 | 37.59 ± 0.23 |
| LPS group | 41.96 ± 0.81 | 46.84 ± 0.57 |
| L + LPS group | 35.70 ± 0.89^{###} | 38.03 ± 0.65^{###} |
| M + LPS group | 36.15 ± 0.45^{###} | 38.15 ± 0.38^{###} |
| H + LPS group | 36.82 ± 0.03^{###} | 38.92 ± 0.71^{###} |
| L group | 33.32 ± 0.67 | 37.00 ± 1.79 |
| M group | 34.26 ± 1.21** | 37.66 ± 0.48 |
| H group | 35.29 ± 0.12*** | 37.93 ± 0.86 |

From Table 3 and FIG. 5 to FIG. 8, it is concluded that in the vetiver experimental group, 62.5 μg/mL, 125 μg/mL, and 250 μg/mL of the water extract of vetivers, alone or together with lipopolysaccharide (LPS), acts on the mouse mononuclear macrophages, and can reduce their NO secretion and iNOS gene expression, showing more significant effects than that of the Radix Astragali control group. This indicates that the drug in Example 2 of the present disclosure can regulate the NO secretion and the expression of iNOS gene in the mouse mononuclear macrophages.

### Use Example 4

The Influence of the Drugs in Example 2 and Comparative Example 1 on the mRNA Relative Expression Levels of Inflammatory Factors of Mouse Mononuclear Macrophages

RAW 264.7 cells (5×10⁵ cells/mL) in the logarithmic growth phase were added to a 12-well plate, and 1 mL of a cell suspension was added to each well. The cell suspension included the RAW 264.7 cells and a cell medium, and the cell medium was a high-sugar DMEM medium containing 10% fetal bovine serum. After culturing for 12 h, a cell supernatant was discarded, and the precipitates were rinsed 2 to 3 times with PBS. The experimental grouping was the same as that in Use Example 1. The diluted drug was added to a 12-well plate, at 1 mL per well, and then incubated in a CO₂ incubator at 37° C. for 48 h. The medium was discarded, and a remaining product was rinsed 2 times with pre-cooled PBS, the cells were collected, and the RNA was extracted, reverse-transcribed, and then detected by fluorescent quantitative PCR. Data processing was conducted using a 2^{-ΔΔCT} method to calculate changes in gene expression levels of each group.

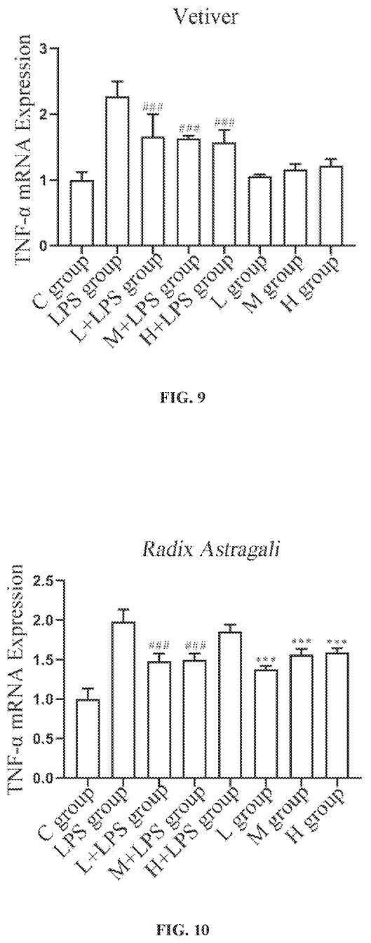

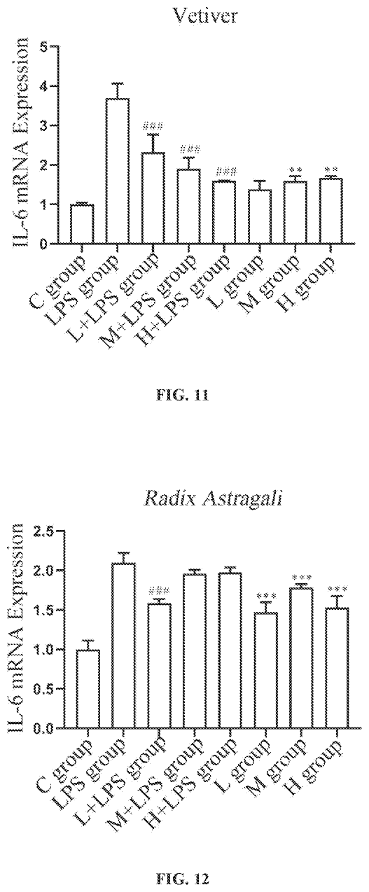

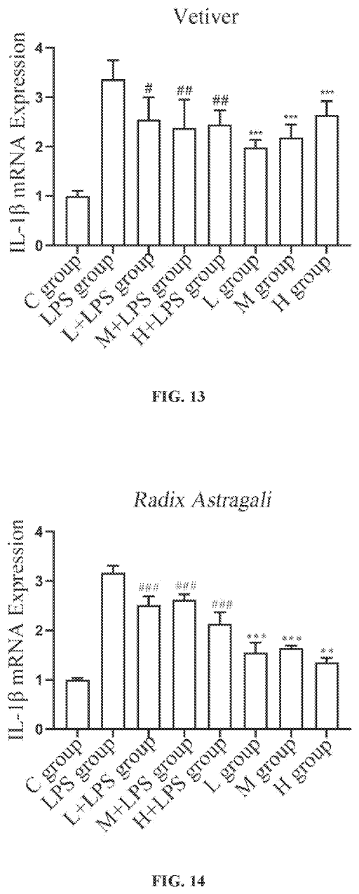

The gene and primer sequences were shown in Table 4, a fluorescent quantitative PCR reaction system was shown in Table 5, and the detection results were shown in FIG. 9 to FIG. 14.

TABLE 4
Gene and primer sequences

| | | | Amplified |
|---|---|---|---|
| | Gene | Primer | fragment |
| Gene | accession | sequence | size |
| name | number | (5′-3′) | (bp) |
| β-actin | NM_007393.5 | F: | 101 |
| | | TACTGC | |
| | | CCTGGC | |
| | | TCCTAG | |
| | | CA | |
| | | (SEQ ID | |
| | | NO: 1) | |
| | | R: | |
| | | TGGACA | |
| | | GTGAGG | |
| | | CCAGGA | |
| | | TAG | |
| | | (SEQ ID | |
| | | NO: 2) | |
| IL-1β | NM_008361.4 | F: | 388 |
| | | TGCAGA | |
| | | GTTCCC | |
| | | CAACTG | |
| | | GTACAT | |
| | | C | |
| | | (SEQ ID | |
| | | NO: 3) | |
| | | R: | |
| | | GTGCTG | |
| | | CCTAAT | |
| | | GTCCCC | |
| | | TTGAAT | |
| | | C | |
| | | (SEQ ID | |
| | | NO: 4) | |
| IL-6 | NM_001314054.1 | F: | 141 |
| | | AAGTGC | |
| | | ATCATC | |
| | | GTTGTT | |
| | | CATACA | |
| | | (SEQ ID | |
| | | NO: 5) | |
| | | R: | |
| | | GAGGAT | |
| | | ACCACT | |
| | | CCCAAC | |
| | | AGACC | |
| | | (SEQ ID | |
| | | NO: 6) | |
| TNF-α | NM_001278601.1 | F: | 250 |
| | | TGGAGT | |
| | | CATTGC | |
| | | TCTGTG | |
| | | AAGGGA | |
| | | (SEQ ID | |
| | | NO: 7) | |
| | | R: | |
| | | AGTCCT | |
| | | TGATGG | |
| | | TGGTGC | |
| | | ATGAGA | |
| | | (SEQ ID | |
| | | NO: 8) | |
| iNOS | NM_001313922.1 | F: | 199 |
| | | TCCTAC | |
| | | ACCACA | |
| | | CCAAAC | |
| | | (SEQ ID | |
| | | NO: 9) | |
| | | R: | |
| | | CTCCAA | |
| | | TCTCTG | |
| | | CCTATC | |
| | | (SEQ ID | |
| | | NO: 10) | |

TABLE 5
Fluorescent quantitative PCR reaction system

| | | |
|---|---|---|
| PCR product | 2 | μL |
| Upstream primer F | 0.4 | μL |
| Downstream primer R | 0.4 | μL |
| Water | 7.2 | μL |
| Luciferase | 10 | μL |
| Total | 20 | μL |

A reaction program adopted a two-step method, including: at 95° C. for 3 min; at 95° C. for 10 sec, at 60° C. for 30 sec; at 95° C. for 10 sec, at 65° C. for 5 sec, at 95° C. for 0.5 sec, conducting a total of 40 cycles. The obtained data were standardized with β-actin as an internal reference, and expressed as a fold difference between the control and treatment groups according to the 2^{-ΔΔCT} method, with three replicates for each group.

From FIG. 9 to FIG. 14, it is concluded that in the present disclosure, the 3 different concentrations of the water extract of vetivers of the vetiver experimental group, and the 3 different concentrations of the Radix Astragali water extracts of the Radix Astragali control group all act on macrophages, alone or together with LPS. The results prove that the above water extracts all could reduce the expression of TNF-α gene, IL-1β gene, and IL-6 gene in mouse mononuclear macrophages, and can significantly alleviate the overexpression of inflammatory factors caused by the LPS group. This shows that the drug in Example 2 of the present disclosure has an effective alleviating effect on inflammation.

### Use Example 5

### The Influence of the Drugs in Example 2 and Comparative Example 1 on Xylene-Induced Mouse Auricle Swelling

Female SPF-grade Kunming mice weighing 18 g±1 g were purchased from Beijing HFK Bio-technology Co., Ltd., with a license number SCXK (Beijing) 2019-0008. The mice were acclimated to the environment for 3 d before the experiment, and then fed with the experimental drugs every day at 9 a.m. The mice were randomly divided into a blank control group, a dexamethasone (DXM) group, and high-, medium-, and low-dose groups of the water extract of vetiver (the water extract of vetiver prepared in Example 2) or high-, medium-, and low-dose groups of the Radix Astragali water extract (the Radix Astragali water extract prepared in Comparative Example 1), with 8 mice in each group. The mice in each group were intragastrically administered 1 time a day for 7 consecutive days. At 1 h after the final administration, 30 μL of xylene was added to the right ear of each mouse in each group, and while the left ear was kept as a control. After 30 min of the treatment, the mice were killed by neck breaking, and round ear pieces were collected by punching at a same sites of the left and right ears with a 6 mm diameter, and the ear swelling degrees and swelling inhibition rates were calculated. The results were shown in Table 6, swelling inhibition rate (%)=(average swelling degree of blank group−average swelling degree of administration group)/average swelling degree of blank group×100%.

TABLE 6
Influence of drugs in Example 2 and Comparative Example 1 on xylene-induced mouse auricle swelling

| | | | Swelling | Swelling |
|---|---|---|---|---|
| Test group | Dosage | | degree (mg) | inhibition rate (%) |
| Blank control group | 0.9% normal | | 5.167 ± 0.896^{###} | — |
| | saline/mice/d | | | |
| DXM group | 0.5 | mg/mouse/d | 2.033 ± 0.907*** | 60.646 |
| water extract of vetiver high-dose group | 74 | mg/mouse/d | 3.067 ± 0.802* | 40.645 |
| water extract of vetiver medium-dose group | 37 | mg/mouse/d | 2.500 ± 0.954** | 51.613 |
| water extract of vetiver low-dose group | 19 | mg/mouse/d | 4.067 ± 1.750^{#} | 21.290 |
| Radix Astragali water extract high-dose group | 74 | mg/mouse/d | 3.700 ± 0.600^{#} | 28.387 |
| Radix Astragali water extract medium-dose | 37 | mg/mouse/d | 3.267 ± 0.153* | 36.774 |
| group | | | | |
| Radix Astragali water extract low-dose group | 19 | mg/mouse/d | 3.467 ± 0.451* | 32.903 |

Notes:
compared with the blank control group,
*P < 0.05,
**P < 0.01,
***P < 0.001; compared with the LPS group,
^{#}P < 0.05,
## P < 0.01,
^{###}P < 0.001 vs LPS group; the same below.

It is concluded from Table 6 that the 3 different doses of water extract of vetiver groups and the 3 different doses of Radix Astragali water extract groups all could reduce auricle swelling to varying degrees and increase the swelling inhibition rates. Compared with the blank control group, the auricle swelling degree of the water extract of vetiver medium-dose group, with a swelling inhibition rate of 51.613%, is similar to that of the DXM group, (swelling inhibition rate of 60.646%), which all show significant differences (P<0.01, P<0.001). However, the anti-inflammation effect of the Radix Astragali water extract medium-dose group is not as desirable as that of the water extract of vetiver medium-dose group, and the swelling inhibition rate is only 36.774%. This shows that the effect of the drug in Example 2 of the present disclosure on xylene-induced ear swelling in mice is significantly better than that of the drug in Comparative Example 1, and a beneficial effect is more significant.

### Use Example 6

### The Influence of the Drugs in Example 2 and Comparative Example 1 on Carrageenan-Induced Mouse Pedal Swelling

Female SPF-grade Kunming mice weighing 18 g±1 g were purchased from the Beijing HFK Bio-technology Co., Ltd., with a license number SCXK (Beijing) 2019-0008. The mice were acclimated to the environment for 3 d before the experiment, and then fed with the experimental drugs. The mice were randomly divided into a blank control group, a DXM group, and high-, medium-, and low-dose groups of the water extract of vetiver (the water extract of vetiver prepared in Example 2) or high-, medium-, and low-dose groups of the Radix Astragali water extract (the Radix Astragali water extract prepared in Comparative Example 1), with 8 mice in each group. The mice in each group were administered continuously for 7 d. At 1 h after the final administration, 1% carrageenan was subcutaneously injected into the sole of the right hind limb to induce inflammation (at 0.1 mL/mouse). At 0 h, 1 h, 2 h, 3 h, and 4 h of Inflammatory treatment, the thickness of the paws of the mice was measured with a micrometer, and the swelling degree and swelling inhibition rate of the paws were calculated. The results were shown in Tables 7 to 8.

TABLE 7
Influence of drugs in Example 2 and Comparative Example
1 on carrageenan-induced mouse pedal swelling degree

| | | | water extract | water extract |
|---|---|---|---|---|
| | Blank | | of vetiver | of vetiver |
| Test | control | | high-dose | medium-dose |
| group | group | DXM group | group | group |
| Dosage | 0.9% normal | 0.5 mg/mouse/d | 74 mg/mouse/d | 37 mg/mouse/d |
| | saline/mice/d | | | |
| 0 h | 1.161 ± 0.121 | 0.995 ± 0.076 | 1.158 ± 0.209 | 1.101 ± 0.159 |
| 1 h | 1.542 ± 0.193 | 1.090 ± 0.134* | 1.161 ± 0.200 | 1.256 ± 0.148 |
| 2 h | 1.666 ± 0.146 | 1.083 ± 0.360** | 1.278 ± 0.083* | 1.311 ± 0.149* |
| 3 h | 1.791 ± 0.121 | 1.328 ± 0.040*** | 1.481 ± 0.081** | 1.588 ± 0.143^{#} |
| 4 h | 1.656 ± 0.059 | 1.438 ± 0.172 | 1.603 ± 0.250 | 1.508 ± 0.057 |

| | water extract | Radix Astragali | Radix Astragali | Radix Astragali |
|---|---|---|---|---|
| | of vetiver | water extract | water extract | water extract |
| Test | low-dose | high-dose | medium-dose | low-dose |
| group | group | group | group | group |
| Dosage | 19 mg/mouse/d | 74 mg/mouse/d | 37 mg/mouse/d | 19 mg/mouse/d |
| 0 h | 1.108 ± 0.140 | 1.059 ± 0.215 | 1.098 ± 0.081 | 1.089 ± 0.096 |
| 1 h | 1.290 ± 0.268 | 1.172 ± 0.439 | 1.363 ± 0.148 | 1.304 ± 0.326 |
| 2 h | 1.354 ± 0.138 | 1.438 ± 0.221^{#} | 1.474 ± 0.036^{#} | 1.550 ± 0.180^{##} |
| 3 h | 1.507 ± 0.188* | 1.586 ± 0.080^{#} | 1.622 ± 0.070^{#} | 1.601 ± 0.162^{#} |
| 4 h | 1.507 ± 0.112 | 1.520 ± 0.068 | 1.514 ± 0.132 | 1.582 ± 0.236 |

TABLE 8
Influence of drugs in Example 2 and Comparative Example 1 on
inhibition rate of carrageenan-induced mouse pedal swelling

| Test group | Dosage | | 0 h (%) | 1 h (%) | 2 h (%) | 3 h (%) | 4 h (%) |
|---|---|---|---|---|---|---|---|
| DXM group | 0.5 | mg/mouse/d | 14.323 | 29.302 | 34.994 | 25.884 | 13.173 |
| water extract of vetiver | 74 | mg/mouse/d | 0.316 | 24.735 | 23.289 | 17.306 | 3.190 |
| high-dose group | | | | | | | |
| water extract of vetiver | 37 | mg/mouse/d | 5.224 | 18.574 | 21.329 | 11.332 | 8.946 |
| medium-dose group | | | | | | | |
| water extract of vetiver | 19 | mg/mouse/d | 4.564 | 16.369 | 18.727 | 15.891 | 8.987 |
| low-dose group | | | | | | | |
| Radix Astragali water | 74 | mg/mouse/d | 8.812 | 23.978 | 13.685 | 11.481 | 8.202 |
| extract high-dose group | | | | | | | |
| Radix Astragali water | 37 | mg/mouse/d | 5.454 | 11.636 | 11.545 | 9.472 | 8.584 |
| extract medium-dose group | | | | | | | |
| Radix Astragali water | 19 | mg/mouse/d | 6.171 | 15.418 | 6.943 | 10.644 | 4.458 |
| extract low-dose group | | | | | | | |

It is seen from Tables 7 to 8 that the mouse pedal swelling in the blank control group is significant, while the other drug administration groups have different degrees of inhibition on the mouse pedal swelling. Compared with the blank control group, at the time point of 1 h to 3 h, the DXM group and the 3 different concentrations of water extract of vetiver groups significantly reduce the degree of carrageenan-induced mouse pedal swelling (P<0.05, P<0.01, and P<0.001), and the water extract of vetiver high-dose group has a better effect. However, although the 3 dosages of Radix Astragali water extract groups have a tendency to alleviate the swelling, the effect is not significant (P>0.05).

### Use Example 7

### The Influence of the Drugs in Example 2 and Comparative Example 1 on an NO Content in Serum of Mice

The mice in each experimental group in Use Example 6 were used as research objects. Blood was collected from eyeballs, serum was separated, stored at −20° C., and level of NO in serum was determined. The specific operation steps and calculation methods were conducted according to the instructions of the NO kit (Beyotime; product number S0021S). The test results were shown in Table 9.

TABLE 9
Influence of drugs in Example 2 and Comparative
Example 1 on NO content in serum of mice

| | | | NO content |
|---|---|---|---|
| Test group | Dosage | | (μmol/mL) |
| Blank control group | 0.9% normal | | 15.351 ± 1.643^{###} |
| | saline/mice/d | | |
| DXM group | 0.5 | mg/mouse/d | 7.737 ± 0.431*** |
| water extract of vetiver | 74 | mg/mouse/d | 8.421 ± 0.672*** |
| high-dose group | | | |
| water extract of vetiver | 37 | mg/mouse/d | 8.404 ± 1.513*** |
| medium-dose group | | | |
| water extract of vetiver | 19 | mg/mouse/d | 9.842 ± 0.518***^{#} |
| low-dose group | | | |
| Radix Astragali water extract | 74 | mg/mouse/d | 13.719 ± 0.895^{###} |
| high-dose group | | | |
| Radix Astragali water extract | 37 | mg/mouse/d | 7.579 ± 1.462*** |
| medium-dose group | | | |
| Radix Astragali water extract | 19 | mg/mouse/d | 6.193 ± 0.249*** |
| low-dose group | | | |

It is shown from Table 9 that: compared with the blank control group, all the drug administration groups, except for the Radix Astragali water extract high-dose one, can significantly reduce the NO levels in the inflammatory tissues of mice (P<0.001). Moreover, the drug obtained in Example 2 of the present disclosure can achieve technical effects equivalent to those of the positive control group of DXM.

### Use Example 8

The Influence of the Drugs in Example 2 and Comparative Example 1 on Relative Expression Levels of Inflammatory Gene mRNAs in Ear Tissue after Inflammation in Mice

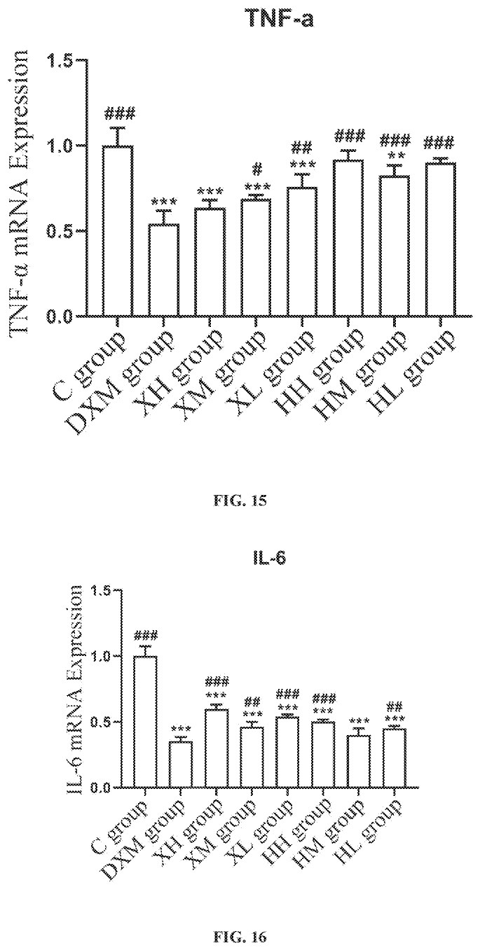

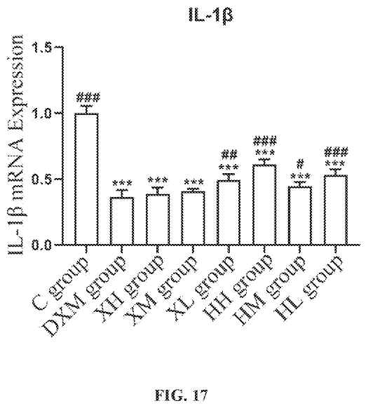

The mice in each group in Use Example 5 were used as research objects. Ear tissues of the mice were cut and ground on ice, total RNA was extracted, and the expression levels of target genes including TNF-α, IL-1β, and IL-6 were detected by fluorescent quantitative RT-PCR. The primer sequences, amplification system, and amplification program were the same as those in Use Example 6, and the detection results were shown in FIG. 15 to FIG. 17. In FIG. 15 to FIG. 17, the C group represented the blank control group, the XH group represented the water extract of vetiver high-dose group, the XM group represented the water extract of vetiver medium-dose group, the XL group represented the water extract of vetiver low-dose group, the HH group represented the Radix Astragali water extract high-dose group, the HM group represented the Radix Astragali water extract medium-dose group, and the HL group represented the Radix Astragali water extract low-dose group.

From FIG. 15 to FIG. 17, it is concluded that, compared with the blank control group, the mRNA expression levels of inflammatory factors TNF-α, IL-1β, and IL-6 are significantly decreased in the mouse inflammatory tissues of the DXM group, as well as in the high-, medium-, and low-dose groups of the water extract of vetiver. The inhibitory effects of TNF-α and IL-1β in the water extract of vetiver high-dose group are basically equivalent to those in the DXM group. However, in spite that the mRNA expression levels of TNF-α, IL-1β, and IL-6 in the mouse samples of the high-, medium-, and low-dose groups of the Radix Astragali water extract also show a downward trend, the effect is not as desirable as that of the water extract of vetiver groups. This fully demonstrates that the drug in Example 2 of the present disclosure has a significant effect of alleviating inflammation.

From the above examples, it is concluded that through acting on mouse mononuclear macrophages and the body, the water extract of vetiver provided by the present disclosure significantly improves the immune regulation function of cells, reduces the expression of various inflammatory factors in cells, and has a significant alleviating effect on inflammation in mice.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A vetiver (Chrysopogon zizanioides) product having an anti-inflammatory effect and capable of improving animal immunity, wherein the vetiver product comprises a water extract of vetiver, the vetiver product is a decoction.

2. The vetiver product according to claim 1, wherein the water extract of vetiver has a concentration of 0.5 g/mL to 2 g/mL.

3. The vetiver product according to claim 1, wherein the vetiver product has the water extract of vetiver with a mass percentage of 1.9% to 100%.

4. The vetiver product according to claim 3, wherein the water extract of vetiver has a concentration of 0.5 g/mL to 2 g/mL.

5. A method for preparing a water extract of vetiver, comprising:
immersing a root of vetiver into water to obtain a immersed solid-liquid mixture, decocting the immersed solid-liquid mixture, and separating the solid and liquid to obtain the water extract of vetiver.

6. The method according to claim 5, wherein the decocting is conducted 1 to 3 times.

7. The method according to claim 5, wherein during the decocting, the root of vetiver and the water are at a mass-to-volume ratio of 1 g:(10-20) mL.

8. The method according to claim 5, wherein the decocting is conducted at 90° C. to 100° C. for 30 min to 60 min each time.

9. The method according to claim 8, wherein the decocting is conducted 1 to 3 times.

10. The method according to claim 8, wherein during the decocting, the root of vetiver and the water are at a mass-to-volume ratio of 1 g:(10-20) mL.

11. A method for improving animal immunity, comprising administering a vetiver (Chrysopogon zizanioides) product to an individual in need thereof.

12. The method according to claim 11, wherein the vetiver product comprises a water extract of vetiver.


SEQUENCE LISTING (10 sequences)

SEQ ID NO: 1 - DNA, 20 bp, synthetic construct
  source 1..20: mol_type = other DNA
tactgccctggctcctagca

SEQ ID NO: 2 - DNA, 21 bp, synthetic construct
  source 1..21: mol_type = other DNA
tggacagtgaggccaggatag

SEQ ID NO: 3 - DNA, 25 bp, synthetic construct
  source 1..25: mol_type = other DNA
tgcagagttccccaactggtacatc

SEQ ID NO: 4 - DNA, 25 bp, synthetic construct
  source 1..25: mol_type = other DNA
gtgctgcctaatgtccccttgaatc

SEQ ID NO: 5 - DNA, 24 bp, synthetic construct
  source 1..24: mol_type = other DNA
aagtgcatcatcgttgttcataca

SEQ ID NO: 6 - DNA, 23 bp, synthetic construct
  source 1..23: mol_type = other DNA
gaggataccactcccaacagacc

SEQ ID NO: 7 - DNA, 24 bp, synthetic construct
  source 1..24: mol_type = other DNA
tggagtcattgctctgtgaaggga

SEQ ID NO: 8 - DNA, 24 bp, synthetic construct
  source 1..24: mol_type = other DNA
agtccttgatggtggtgcatgaga

SEQ ID NO: 9 - DNA, 18 bp, synthetic construct
  source 1..18: mol_type = other DNA
tcctacaccacaccaaac

SEQ ID NO: 10 - DNA, 18 bp, synthetic construct
  source 1..18: mol_type = other DNA
ctccaatctctgcctatc